(19) United States
(12) Patent Application Publication
(10) Pub. No.: US 2023/0031710 A1
(43) Pub. Date: Feb. 2, 2023

(54) SYSTEMS AND METHOD FOR CONTROLLING FLUID FLOW IN BIOREACTORS

(71) Applicant: CYTIVA SWEDEN AB, Uppsala (SE)

(72) Inventors: Praveen Paul, Hoodi Village, Bengaluru (IN); Sahebagouda Alagur, Hoodi Village, Bengaluru (IN); Thomas Falkman, Uppsala (SE); Prashanth Hosabettu Mohan, Hoodi Village, Bengaluru (IN)

(21) Appl. No.: 17/782,591

(22) PCT Filed: Dec. 17, 2020
(86) PCT No.: PCT/EP2020/086853

§ 371 Date: Jun. 3, 2022

(30) Foreign Application Priority Data
Jan. 7, 2020 (IN) 202011000693

(51) Int. Cl. C12M 1/00; C12M 1/34

(52) U.S. Cl. CPC C12M 29/10; C12M 29/16; C12M 41/40

(57) ABSTRACT

Systems (700) and methods (800) for method of continuous fluid flow in a bioreactor (700) is provided. The method (800) comprises providing (805) a bioreactor system (700) including a bioreactor volume (720), a filtration part (730), and a recirculation line (721) including a recirculation pump (722) is provided between the bioreactor volume (720) and the filtration part (730). The method (800) further comprises providing (810) a plurality of sensors (760) along the recirculation line (721) and monitoring the fluid flow parameters using the sensors (760). The method further comprises sending (820) a plurality of signals from the sensors (760) indicative of the fluid flow parameters to one or more controllers; and controlling (830) the fluid flow rate at the recirculation pump (722) by means of the or each controller.

### FIELD OF THE INVENTION

Embodiments of the present specification relate generally to systems and methods of continuous fluid flow in bioreactors and more specifically to systems and methods for automated continuous fluid flow in bioreactors.

### BACKGROUND OF THE INVENTION

Bioreactors are widely in used for biomanufacturing of biotechnology products. Several varieties of bioreactors are currently available in the market that process organisms, chemicals, nutrients etc. based on the desired qualities of the biotechnology product. Process parameters of the reactants within the bioreactor directly affect the quality of the product. Some typical process parameters of the substrates within the bioreactor are pH, temperature of the cell culture, glucose, oxygen levels, conductivity, colour change etc. These reactants may be fed to the bioreactor at once and processed in what is well-known as “batch processing”. Alternatively, these reactants are continuously fed to the bioreactor in “continuous processing”. Perfusion is a process through which the yield of a cell culture is improved by continuous removal of used media or products from the bioreactor and addition of fresh media. Perfusion is getting attention of the biopharma manufactures as a part of the continuous-manufacturing. In perfusion processes, the product is continuously harvested from the bioreactor while new reaction media is fed into the bioreactor. While batch processes last for few hours or days, perfusion processes may go on for weeks or months.

When cells/organisms, nutrients and chemicals are fed inside the bioreactor and desired process parameters are maintained, cell growth starts within the bioreactor. Cell growth may include increase in number of cells by multiplication of cells or growth in physical parameters of individual cells. Continuous feeding of media, increase in number of cells and increase in weight of individual cell collectively increases the weight of the bioreactor. If the weight of the bioreactor increases beyond the maximum designated threshold capacity of the bioreactor, bioreactor performance in terms of quality of cells, uniformity of the cell output, process parameters of the reactants etc. is adversely affected. Accordingly, in traditional bioreactors, there is a provision of a filter and a permeate line to drain out cell-media mixture from the bioreactor corresponding to the weight of the inputted media.

Traditional systems operate on the principle of “volume-in, volume-out”, meaning the volume (ml) of the media fed to the bioreactor is equal to the volume (ml) of the content drained out by the motor pump from the bioreactor. Continuous feeding of media and perfusion of proportionate amount of cell culture out of the bioreactor has several drawbacks. Continuous perfusion and collection of permeate leads to deposition of cells in the filter. Filter clogging leads to reduced output from the filter. In the event of clogging of the filter, to maintain uniform rate of permeate flowing out from the filter, speed of the motor pump is required to be increased. This leads to excess load on the motor pump and increased power consumption. Clogged filter requires timely cleanup to maintain the filter performance. This increases downtime of the filter and the bioreactor.

Additionally, continuous operation of the motor pump increases power consumption and reduces motor life. Traditional motor pump is operated at a definite speed to drain out definite amount of the reaction fluid from the bioreactor without regard to the stage of development of the biological elements within the reactor. This has undesirable effects on development of biological elements. Cell retention systems have been developed to retain the cells within the bioreactor and let only the media go out of the bioreactor. However, there is additional cost associated with these systems. Accordingly, current approaches to perfusion suffer from many disadvantages. Equipment suppliers in biotechnology industry need to respond with more durable, efficient bioreactors with different sensors and monitoring technologies that can be integrated with the existing bioreactors without significantly altering the hardware connections in the system.

### BRIEF DESCRIPTION OF THE INVENTION

In accordance with one aspect of the invention a perfusion control system for a bioreactor is disclosed. The system comprises a media container adapted to store reaction media and a weighing scale configured to measure the weight of the media container. The bioreactor is connected to the media container through a media feed line and a motor pump is provided to continuously feed the media from the media container to the bioreactor. A weighing scale is provided to measure the weight of the bioreactor. Further, a plurality of filters are connected to the bioreactor through a recirculation line. A recirculation motor pump is provided on the recirculation line to transfer the reaction fluid from the bioreactor to the filter. A plurality of sensors are provided on the recirculation line to measure the process parameters of the fluid and provide a feedback signal to control the process parameters of the fluid.

In accordance with another aspect of the invention a method of continuous fluid flow in a bioreactor is provided. The method comprises providing a bioreactor system including a bioreactor volume, a filtration part, and a recirculation line including a recirculation pump is provided between the bioreactor volume and the filtration part. The method further includes providing continuous media feed to the bioreactor at a user determined rate and operate the recirculation pump at a user determined rate. The method further comprises measuring the fluid flow parameters using a plurality of sensors along the recirculation line and providing a feedback to control the recirculation pump operating parameters to maintain the continuous flow of the fluid.

The above advantages and other advantages and feature of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this specification.

### BRIEF DESCRIPTION OF THE DRAWINGS OF THE INVENTION

These and other features of the embodiments of the present specification will be better understood when the following non-limiting embodiments in the detailed description are read with reference to the accompanying drawings, wherein below:

FIG. 1 illustrates a perfusion control system in accordance with aspects of the present specification.

FIG. 2 is a detailed view of the perfusion control system of FIG. 1, in accordance with aspects of the present specification.

FIG. 3(a)-3(b) is a detailed view of the flow control process of the media pump in accordance with aspects of the present specification.

FIG. 4(a)-4(b) illustrate an independent movable support integrated with the bioreactor.

FIG. 4(C) illustrates independently moveable support with a user interface.

FIG. 5 illustrates one approach of controlling the perfusion in bioreactor.

FIG. 6 illustrates another approach of controlling the perfusion in bioreactor.

FIG. 7 illustrates a system in accordance with further aspects of the present specification.

FIG. 8 illustrates a method in accordance with further aspects of the present specification.

### DETAILED DESCRIPTION

The following detailed description of the exemplary embodiments refers to the accompanying drawings. The same reference numbers in different drawings identify the same or similar elements. Additionally, the drawings are not necessarily drawn to scale. Also, the following detailed description does not limit the invention. Instead, the scope of the invention is defined by the appended claims.

Reference throughout the specification to “one embodiment” or “another embodiment” or “some embodiments” means that the particular feature, structure or characteristic described in connection with an embodiment is included in at least one embodiment of the subject matter disclosed. Thus, the appearance of the phrase “in one embodiment” or “in an embodiment” or “in some embodiments” in various places throughout the specification is not necessarily referring to the same embodiment(s). Further, the particular features, structures or characteristics may be combined in any suitable manner in one or more embodiments.

Bioreactors are specially manufactured systems or vessels used in biotechnology industry for carrying out various processes that use variety of chemicals, organisms, nutrients and substances derived therefrom that together constitute “process fluid”. Bioreactors are typically used to grow cell cultures using aerobic or anerobic processes in generally cylindrical bioreactor vessels.

Manufacturing biotechnology products using bioreactors include preparation of raw material in upstream processing. The raw material may be biological or non-biological in origin. This raw material along with the other reactants is fed into the bioreactor to carry out controlled processing of the reactants. Several process parameters are adjusted and controlled to impart desired qualities to the product. Perfusion is a process where the product or the process fluid is continuously harvested from the bioreactor while new media is fed. Motor pumps are employed to harvest the product from the bioreactor. These motor pumps can be configured to output the product or reaction fluid based on the input weight of the media. Recirculation of the process fluid is carried out using one or more motor pumps, filters, valves, pressure retentate and pressure permeate. Dead cells, excess fluid and other waste material is separated from the product and drained out. Part of the process fluid that requires further processing is recirculated through the bioreactor. A media feed line is provided to feed the fresh media into the bioreactor from the media container.

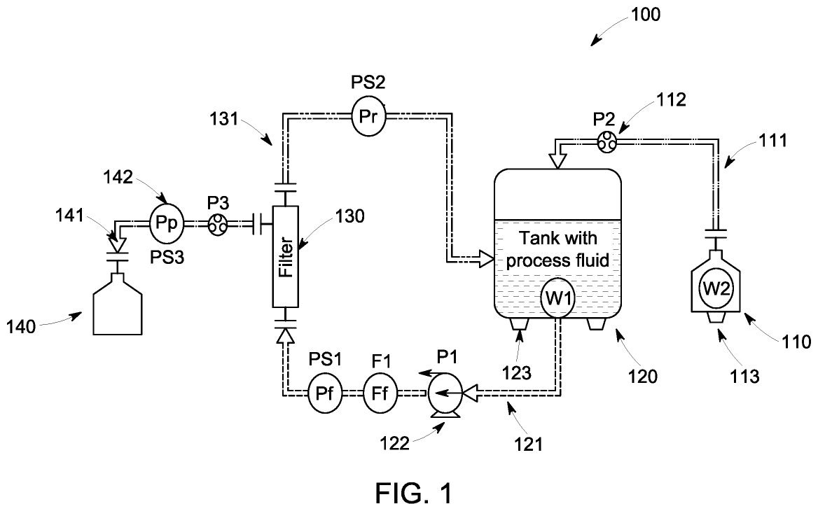

Referring to FIG. 1, a schematic representation of the bioreactor (120) and perfusion system (100) in accordance with an embodiment of the present application. The reaction media is contained within the container (110) and the container (110) is connected to bioreactor (120) using a media feed line (111). A motor pump (112) is provided on the media feed line (111) for transferring the media from the container (110) to the bioreactor (120). The motor pump (112) may be a peristaltic pump, however, any other kind of suitable motor pump may be employed to transfer the media from the container (110) to the bioreactor (120).

A traditional or electronic weighing scale (113) is provided to continuously measure the weight of the container (110). Similarly, a weighing scale (123) is provided to measure the weight of the bioreactor vessel (120). When media is transferred from the container (110) to the bioreactor (120), there is reduction in weight for the container (110) and gain the weight of the bioreactor (120) equal to the weight of the reaction media transferred. Accordingly, gain in the weight of the bioreactor (120) is monitored to control the process parameters of the reaction fluid within the bioreactor (120).

This feed to the bioreactor (120) is fixed at a user set flow rate. Depending on the viable cell density within the bioreactor (120), a cell specific perfusion rate (CSPR) is determined. Alternatively, amount of vessel volume per day (VVD) feed to the bioreactor (120) is determined and the motor pump (112) is configured to input the VVD amount into the bioreactor (120).

According to an embodiment of the present specification, the weight (W) of the bioreactor (120) varies within upper weight limit (U) and lower weight limit (L) of the bioreactor (120). This upper weight limit (U) and lower weight limit (L) may be predetermined for efficient control of the weight (W) of the bioreactor (120). For example, if one percent (1%) weight band is decided for the bioreactor (120), the upper weight limit (U) will be (W+0.5% of W) and lower weight limit (L) will be (W−0.5% of W). As the media is fed into the bioreactor (120), weight (W) of the bioreactor (120) starts rising towards upper weight limit (U). The weighing scale (123) measures the weight of the bioreactor (120).

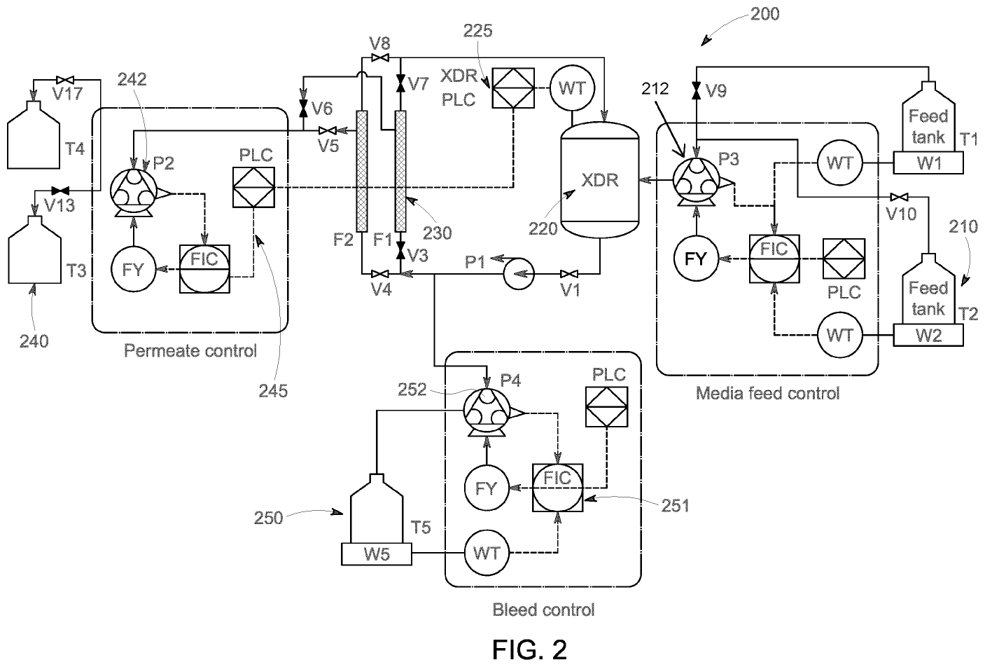

A filter (130) is connected to the bioreactor (120) using a recirculation line (121) and a motor pump (122) is provided on the recirculation line (121) for exchange of reaction fluid within the bioreactor (120) to the filter (130). A controller (shown in FIG. 2) is connected to the weighing scale (123) for receiving the signals representative of the weight (W) of the bioreactor (120) and transmit the signal to motor pump (122). The controller is also configured to receive signals from the motor pump (112) indicative of the media feed to the bioreactor (120).

The filter (130) is connected to the bioreactor using a retentate line (131). The filter (130) is further connected to a permeate tank (140) through a permeate line (141). A motor pump (142) is provided on the permeate line (141) to transfer the permeate from the filter (130) to the permeate tank (140). Although only one exemplary filter (130) is shown in FIG. 1, a greater number of filters (130) may be used based on the quantity of the process fluid.

The motor pump (142) is connected to a controller that receives signals from the controller connected to the weighing scale (123). The controller connected to the motor pump (142) is configured to operate the motor pump (142) to maintain steady weight (W) of the bioreactor (120).

When weight (W) of the bioreactor (120) goes beyond the upper limit (U) determined for the bioreactor (120), the weighing scale (123) generates a signal corresponding to the current weight (W_{current}) of the bioreactor (120). This signal is transferred to the controller connected to the motor pump (142). The controller operates the motor pump (142) to flow out the permeate from the filter (130) and thereby reduces the total amount of the fluid present in the bioreactor (120). This process continues until the weight (W) of the bioreactor (120) comes down to the predetermined range of for example (U=W+0.5% of W). Once the weight (W) of the bioreactor (120) goes below the maximum upper limit (U) defined for the bioreactor (120), corresponding signal is sent to the controller connected to the motor pump (142) to stop the perfusion of the permeate. This helps in maintaining the weight (W) of the bioreactor within the predetermined range. If the weight (W) of the bioreactor (120) goes down beyond the lower weight limit (L) of the bioreactor (120), then the permeate flow is immediately stopped to once again maintain the weight (W) within the predetermined range. In one example, when weight (W_{current}) of the bioreactor goes beyond the upper weight limit (U), the permeate pump (142) will be operated at double the speed (2X) of the perfusion feed in flow rate, and when weight (W_{current}) of the bioreactor is less than the lower weight limit (L), the permeate pump (142) will continue running at lower than the critical flux of the filter/membrane in usage.

As the permeate flows to the permeate tank (140), retentate is transferred from the filter (130) to bioreactor (120) using the motor pump (122). If weight (W_{current}) of the bioreactor (120) is less than the upper weight limit (U), the retentate may be added to the bioreactor (120) from the filter (130). Alternatively, fresh media may be added to the bioreactor (120) from the container (110) based on the weight (W_{current}) of the bioreactor (120) and cell density in the bioreactor (120). Different sensors may be employed to measure the cell density within the bioreactor (120) to decide the amount of media or amount of retentate to be added to the bioreactor (120).

If weight (Wcurrent) of the bioreactor (120) is less than the upper weight limit (U), the retentate may be added to the bioreactor (120) from the filter (130). Alternatively, fresh media may be added to the bioreactor (120) from the container (110) based on the weight (Wcurrent) of the bioreactor (120) and cell density in the bioreactor (120). Different sensors may be employed to measure the cell density within the bioreactor (120) to decide the amount of media or amount of retentate to be added to the bioreactor (120).

Flow control mechanism illustrated above is triggered by the weight (W) of the bioreactor (120). Such control enables maintaining the weight (W) of the bioreactor (120) within the user determined range. Further, the permeate pump (142) is operated only when the weight of the bioreactor is beyond the permissible upper weight limit (U) and this intermittent operation of the permeate pump (142) saves more power and prolongs working life of the motor pump (142). Intermittent operation of the motor pump (142) enables intermittent cleaning of the filter (130) and system downtime for filter cleaning is saved. Accordingly, there is substantial improvement in filter (130) life and quality. No regard to cell density was given in the traditional volume flow-based systems and good quality cells were lost along with the dead cells during permeate flow. However, according to an embodiment of the instant application, the cell density control is better achieved using the permeate pump (142) that operates based on the weight (W) range (U-L) of the bioreactor (120). Accordingly, the purpose of the perfusion control to maintain a constant feed rate (user defined rate based on VVD or CSPR) to the bioreactor (120) through media feed pump (112) and at the same time to keep the bioreactor weight (W) at steady state by controlling the permeate pump (142) is achieved.

Cell bleed is used in perfusion process to maintain steady state perfusion control and improve the overall cell culture viability. In another embodiment of the present application, if cell bleed control is enabled keeping the media feed rate constant, the change will be on the permeate control to maintain the weight (W) of the bioreactor (120) at steady state. In perfusion process, only spent media is removed and cells are retained by a membrane to eventually increase the cell mass. To over come the effect of nutrients limitation at high cell density that will impact product quality and cell productivity, such high cell density may require higher input of fresh media. Cell bleed is a necessary step to maintain cell viability to control steady state of the process.

FIG. 2 illustrates details of the perfusion control system of FIG. 1. More than one media feed tanks (210) may be employed to ensure supply of the media to the bioreactor (220) at predetermined flow rate. Weighing scales (W₁ and W₂) are employed to continuously monitor the weights of the media tanks (210). Although, only two media tanks are shown in FIG. 2, it is within the scope of the present application to use more than two media tanks (210). A fluid integrated circuit (FIC) is connected to a programmable logic controller and configured to receive the weighing scale signals indicative of the weight of the media feed tank (210). Based on the output of the fluid integrated circuit (FIC), the motor pump (212) is operated to transfer the media from the media feed tank (210) to the bioreactor (220). Filters (230) are connected to the bioreactor (220) through a recirculation line. Although, only two filters are shown in FIG. 2, it is within scope of the present application to use more than two filters for processing the reaction fluid.

Multiple permeate tanks (240) are incorporated to collect the permeate flowing out of the filters (230). A weighing scale measures weight (W) of the bioreactor and a programmable logic controller (PLC) (225) is continuously updated with the weight (W_{current}) of the bioreactor. Another programmable logic controller (PLC) (245) is located closer to the permeate motor pump and receives weight (W_{current}) of the bioreactor. The programmable logic controllers (225, 245) are programmed to operate the permeate motor pump (242) to let out only the used reaction fluid from the filters (230). Cells that are retained by the filter (230) for recirculation are fed back to the bioreactor (220).

Additionally, a cell bleed tank (250) may be employed along with a control unit to monitor the cell bleed. The cell bleed control consists of using a weighing scale to measure the weight of the bleed tank and timely feeding the cell bleed tank (250) in controlled manner. A controller (251) is connected to the cell bleed weighing scale and receives signal indicative of the weight of the cell bleed tank (250). The controller (251) of the cell bleed tank (250) is also connected to the programmable logic controller (245) of the permeate motor pump (242). When the bleed control is also enabled keeping the feed rate of media constant, the change will be on the permeate control (245) to maintain the weight of the bioreactor (220) at steady state. A flow factor is calculated at regular interval for the media feed pump using the weighing scale so the net media feed into the bioreactor (220) is accurate. There are many advantages of calculating the flow factor at regular interval. Pump calibration is not required when flow factor is calculated. Also, wear and tear of the pump tubing over a time will not impact on the perfusion process and feed totalizer accuracy can be maintained. This is based on the continuous monitoring of the cell mass using viable cell density (VCD) sensor or by manually removing some percentage of working volume of bioreactor. In either scenario, based on feedback from the cell density sensor positioned inside the bioreactor (220) or by means of inputting a value manually through a user interface, cells are harvested continuously from the bioreactor (220) to maintain steady state. A control software is provided that contains a code to operate various motor pumps. During the perfusion process, viability cell density (VCD) higher limit value is initially fed into the software. Viability cell density (VCD) value in the bioreactor (220) is monitored continuously by means of a VCD sensor, and if the cell density is more than the set value, then the sensor will send feedback to software which in-turn starts the bleed pump (252) such that it will be harvested continuously until constant viable cell density comes back to initial set value. Once the cell density is within the defined set value the motor pump (242) will stop.

Following example shows specification of the components used in the perfusion process and their operating parameters:

Motor pump: Watson Marlow peristaltic 313 High speed pump (350 rpm)
Weighing scale: 300 kg Weighing scale from METTLER TOLEDO with IND570 Weighing Terminal
Flow rates for different tubing sizes:

Flow rates (ml/min)

| | 1.6 mm (1/16″) wall tubing | | | | | | |
|---|---|---|---|---|---|---|---|
| | 0.5 mm | 0.8 mm | 1.6 mm | 3.2 mm | 4.8 mm | 6.4 mm | 8.0 mm |
| rpm | 1/50″ | 1/32″ | 1/16″ | 1/32″ | 3/16″ | 1/16″ | 5/16″ |
| 100 | 3.00 | 6.00 | 26.0 | 100 | 220 | 360 | 500 |
| 350 | 10.5 | 21.0 | 91.0 | 350 | 770 | 1260 | 1750 |

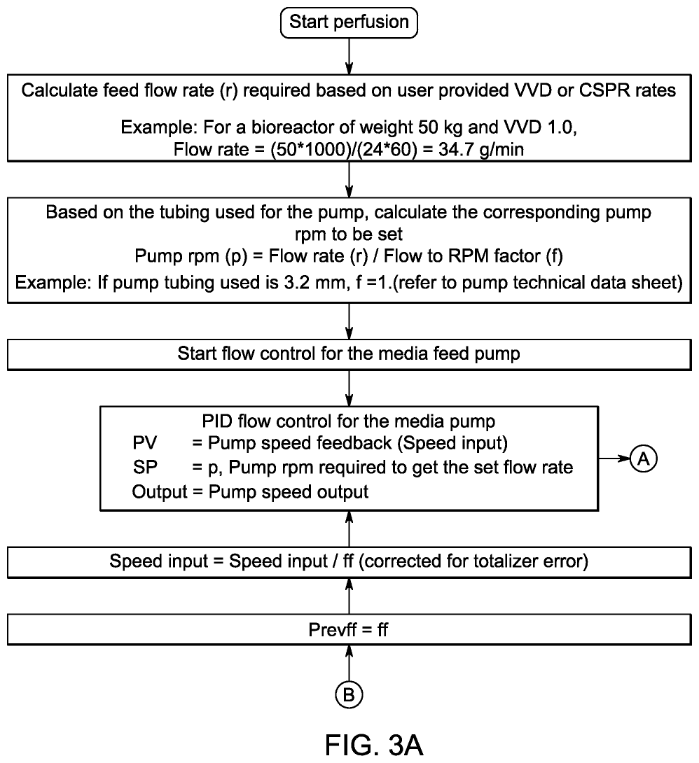

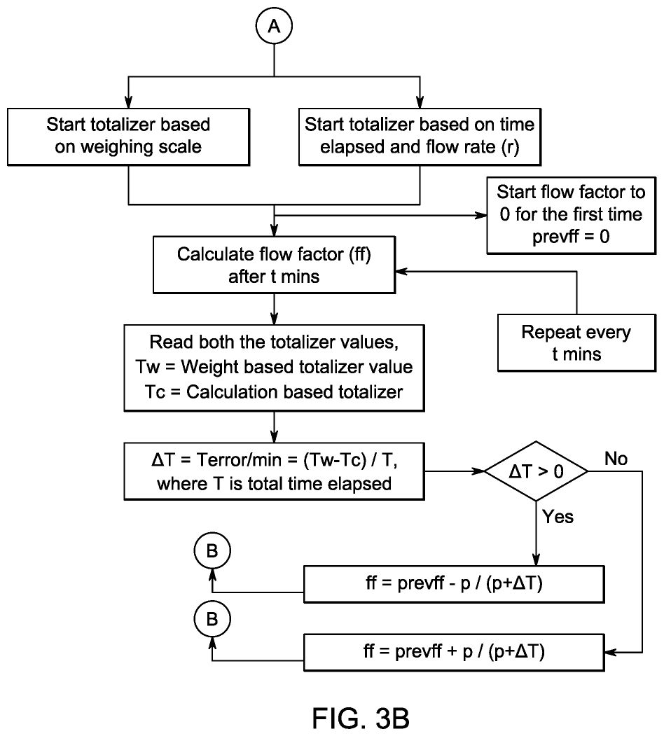

FIGS. 3(a)-3(b) show a flow chart of the media flow control portion (300) of the perfusion process control. Once the perfusion is started (310), the feed flow rate of the media is calculated to determine the amount of media that is required to be fed to the bioreactor (220). For example, if weight of the bioreactor is 50 kilograms and user defined vessel volume per day (VVD) that is fed to the bioreactor is 1, the flow rate of the media is calculated by following calculation:

$$Flow rate = \frac{50 \times 1000}{24 \times 60} = 34.7 grams per min$$

Further, based on the tubing used, pump speed (rpm) is determined (320) by following formula:

$$Pump speed = \frac{Flow rate ( r )}{Flow to RPM factor ( f )}$$

Based on above calculations, media feed motor pump is controlled (340). A PID flow controller is implemented (350) to control the media feed pump. A first totalizer is started (360) based on the weight of the media tank and a second totalizer is started based on the time elapsed from starting the media feed and flow rate of the media, a flow factor (ff) is continuously calculated after specific time (t minutes). This calculation of flow factor (ff) is repeated to identify any errors present in the totalizer. For example, difference in the totalizer values (ΔT) of weight-based totalizer (T_{w}) value and calculation-based totalizer value (T_{c}) is calculated to determine presence of any error and inputted to PID flow control of the media pump. Continuity in media feed is achieved using the method (300) illustrated in FIGS. 3(a)-3(b).

Above process ensures accurate perfusion feed in at constant rate to provide robust control on the perfusion process which would result in the better product quality and improved product titer. Further, various controls enable steady state perfusion process for longer duration. The periodic ON and OFF of permeate motor pump as mentioned in embodiment of FIG. 1 or the periodic change in the permeate flow as mentioned in the embodiment of FIG. 2 improves the filter performance in terms of longevity and usage. Accurate steady state perfusion control without need of accurate scales and using periodic autocorrection of errors and using low accuracy flow sensors is possible with above discussed systems and methods.

Application of continuous manufacturing in biopharmaceutical manufacturing has progressed in the past decade. The conversion of batch processes to continuous manufacturing is the future of the biopharmaceutical industry, and includes employing the continuous flow, end-to-end integration of manufacturing sub-processes with a significant level of control strategies. Continuous biopharmaceutical manufacturing is more time-efficient, reduces energy needs, helps to increase productivity and reduces the amount of overall waste. The risk of human error is also reduced because continuous processing means fewer people are involved in the production process from start to finish.

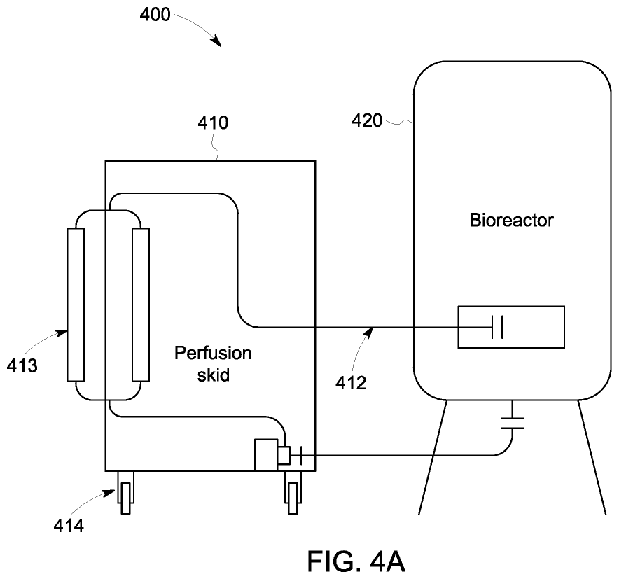

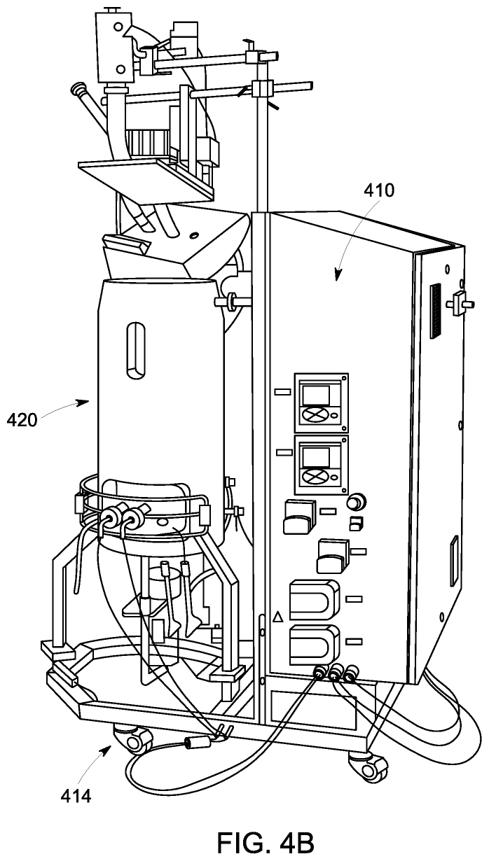

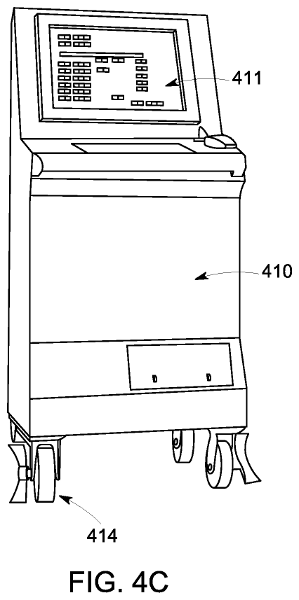

FIG. 4 (a)-4 (b) illustrates integration (400) of perfusion system with the bioreactor. In one embodiment of the present application, the perfusion system of FIGS. 1-2 is provided as a standalone independently moveable support (410) that may be readily integrated with the existing bioreactors (420). The independent movable support (410) includes a computer system having a processor, memory and display screen. The processor is configured to acquire perfusion data and display over the display screen (411) of the user console. A control algorithm is provided in the computer system that allows user of the system to control the perfusion parameters by inputting commands over the display screen (411) of the user console. The filters (413) are connected to the bioreactor (420) through a retentate line (412). Integration of independent movable support with the bioreactor has several advantages including minimum flow-path length to reduce retention time, minimum back pressure through optimized tube sizing, Optimized tubing diameter for pump inlet for minimal air bubble entry into pump, optimum pump location & orientation for natural priming and performance, reduced shear on cells through avoidance of sharp bends in flow-path and minimum number of connections with bioreactor bag.

The independent movable support (410) of the present application may be integrated in “plug and play” format with the bioreactor (420). Plug and play type of flowpaths enable quick integration between the independent movable support (410) and the bioreactor (420) using aseptic connectors. A single user interface and data logging for bioreactor (420) and independent movable support (410) may be provided for efficiently operating the system. A bottom inlet port with larger tubing diameter from bioreactor to independent movable support (410) enables easy liquid flow and avoids bubble entry into the flowkit. Integration of bleed circuit in the retentate flowpath section ensures the stressed cells/concentrated cells. Flowpath can accommodate wide range of filters with different path lengths and single port recovery through independent movable support is possible. Sterile air inlets are provided to enable integrity check in the assembled condition of flowpath and automatic switching of perfusion media and permeate bins to ensure continuous operation.

FIG. 4 (c) shows standalone independent movable support (410) with a user interface (411). The user interface (411) is used to insert process parameters of the bioreactor (420) and process the reaction fluid at a predetermined flow rate. The independent movable support (410) is a wheeled support (414), independently moveable with respect to the bioreactor with flexible sealed fluidic conduit interconnections between the bioreactor (420) and the independent movable support (410).

The independent movable support enables users to maximize their yield in the cell culture in bioreactor. The perfusion independent movable support is essentially a tangential flow filtration system with hollow fibre filters. The system flowpath can be connected with the bioreactor bag. When the user faces clogging of the filter, it is difficult to put a new filter in the flowpath. Integration of perfusion independent movable support enables automated switching to a different filter. Running a perfusion independent movable support needs proper integration with the bioreactor controls. An integrated control of XDR bioreactor and operations on the perfusion independent movable support is provided through the monitoring station screen and no time is needed in customizing the existing systems. All the run data will be saved in the common database with Bioreactor.

The same instrument can be used for different bioreactor sizes and volumes. Flowpath components and filters can be configured for different working volumes and flowrates. Accordingly, users can select the exact tubings based on their application. Further, there is no need to do recirculation pump priming. The location of the pump is provided in such a way that the recirculation pump get naturally primed. All connections are made with Aseptic connections and possibility of contamination of cell culture media is reduced.

Accordingly, integration of perfusion independent movable support with the bioreactor provides automatic switching of perfusion media and permeate. An integrated control of bioreactor and perfusion independent movable support is achieved minimum or no manual intervention is required for filter change.

The steady state perfusion control requirement (the steady state perfusion process) in system is built on the constant (steady) XDR weight. In this requirement, perfusion media addition is tightly controlled and accurate, whereas permeate harvest is controlled to maintain a steady XDR weight.

The system would have a weight-based control for:

1. Perfusion media addition

2. Cell bleed

3. Steady state bioreactor weight

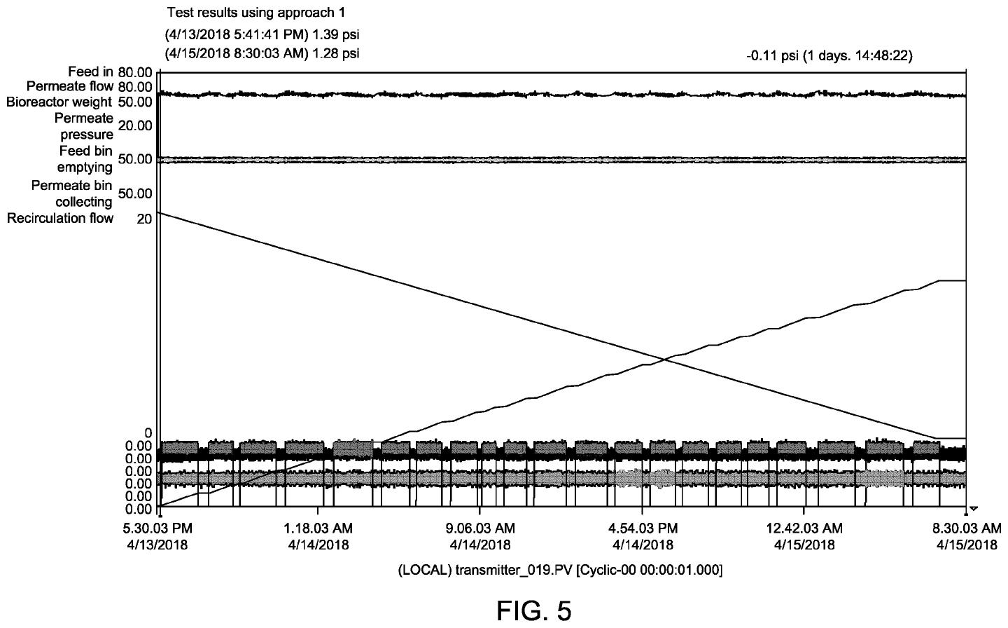

As shown in FIG. 5, in one approach the user can set the flow rate for the perfusion media either based on the metabolic requirements of the cells or based on a volumetric exchange per day. If the process, requires cell bleeding, user can also set a flow rate for the cell bleed. The flow rate for the permeate out is controlled to ensure that the bioreactor weight is maintained steady. For example, the bioreactor (XDR) steady weight is set at 47 kilos. The perfusion media addition is set at 10 ml/min. The bioreactor (XDR) weight is allowed to vary between +200 gm, when bioreactor (XDR) weight crosses 47.2 kgs, the permeate flow rate is set at 1.1 times that of perfusion media addition and again when bioreactor (XDR) weight reaches 47 or 46.8 kg the permeate flow rate set to zero lpm. This approach ensures the bioreactor (XDR) steady weight is maintained at 47±0.2 kg. This approach is on/off control of permeate harvest to maintain the steady bioreactor (XDR) weight.

| | | Steady State |
|---|---|---|
| Perfusion media Addition | Cell bleed | Bioreactor Weight |
| X ml/min | OFF | Maintained by |
| (User configurable) | | controlling permeate |
| | | pump rate equivalent |
| | | to X ml/min |
| X ml/min | Y ml/min | Maintained by |
| (User configurable) | (User configurable) | controlling permeate |
| | | pump rate equivalent |
| | | to X-Y ml/min |

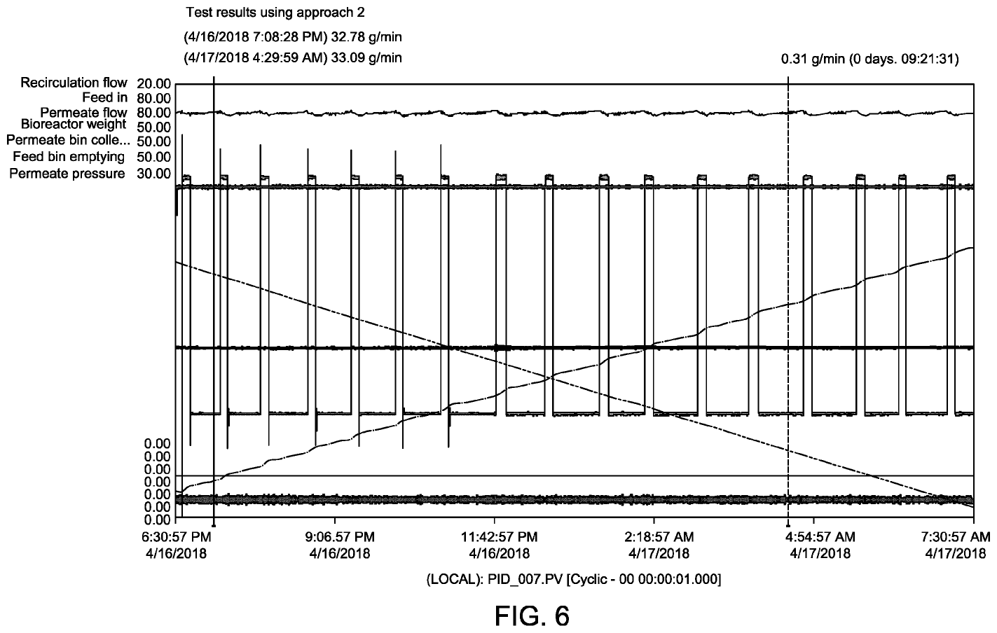

As shown in FIG. 6, in another approach that differs from previous approach in the way the permeate pump is operated when an increase in the bioreactor (XDR) weight is detected. In this approach, the user has an option to set high and low limits for the permeate pump rate. The permeate pump would then run at the set low limit till a change in the bioreactor weight is detected, after which it runs at the set high limit till the bioreactor weight reaches the set point for the steady bioreactor weight. User has an option to set the lower limit of the permeate pump rate to zero if the user prefers an intermittent ON/OFF permeate flow which could enhance the HFF membrane performance compared to a constant permeate out of the HFF membrane.

In the trends shown in the second graph, the bioreactor (XR) weight is set at 47 kgs, and perfusion media addition rate at 33 ml/min, which is constant and accurate. The permeate harvest flowrate set at 24 ml/min. When bioreactor (XXR) weight crosses ±200 gm i.e. 47.2 kg, the permeate flow rate is increased to double (2×) of perfusion media addition. This is again to maintain the steady XDR weight, however the permeate harvest is allowed to switch between two flow rates, which is again user configurable. By allowing permeate flowrate to vary, permeate back pressure is provided which could improve filter performance over the period.

| | | Steady State |
|---|---|---|
| Perfusion media Addition | Cell bleed | Bioreactor Weight |
| X ml/min | OFF | Maintained by setting |
| (User configurable) | | permeate pump rate to |
| | | run at two set points |
| | | (User configurable) |
| X ml/min | Y ml/min | Maintained by setting |
| (User configurable) | (User configurable) | permeate pump rate to |
| | | run at two set points |
| | | (User configurable) |

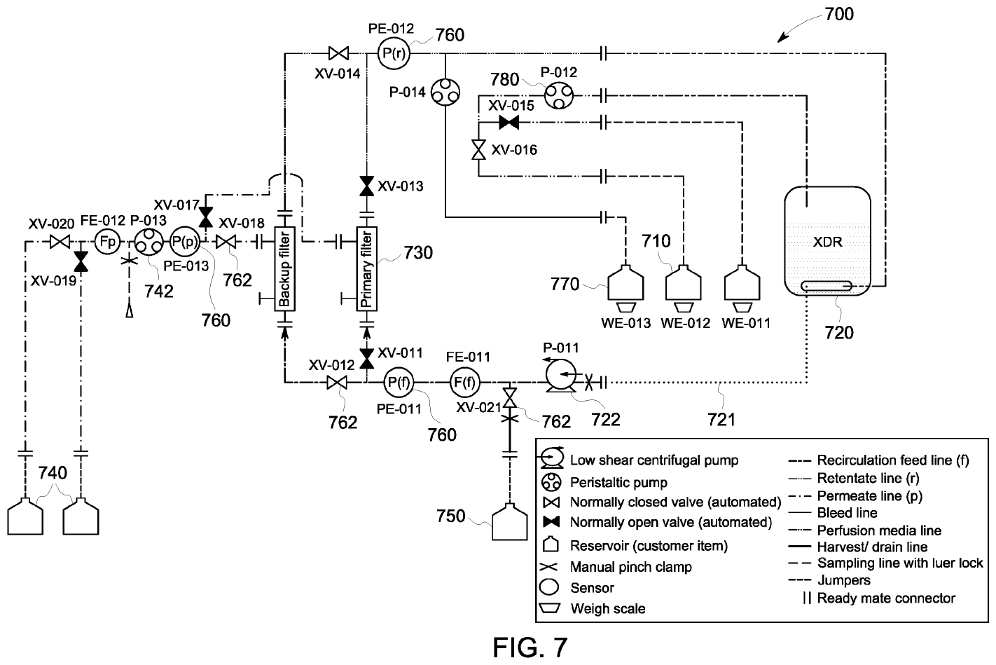

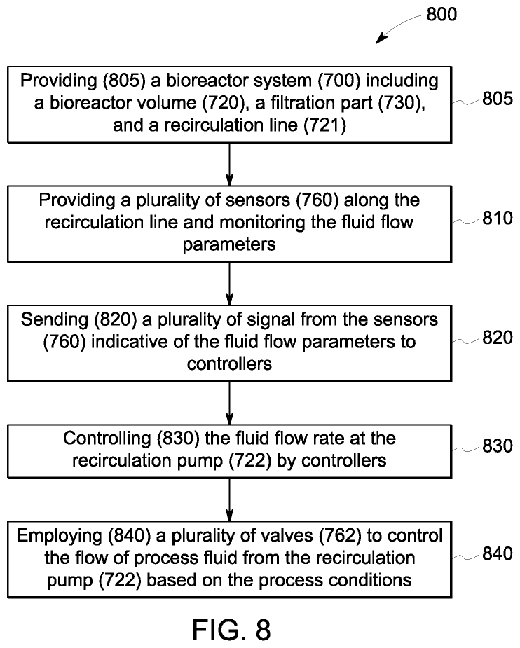

FIG. 7 illustrates a system (700) similar to the system of FIGS. (1)-(2). Additionally, sensors (760) are incorporated along the recirculation line (721). The sensors (760) are preferably single use pressure sensors and monitor the process pressure in the flowpath. Fluid flow parameters like transmembrane pressure (TMP), Pressure difference (Delta P) could be derived from these sensor values. This flow sensors monitor the recirculation flowrate. Sensors (760) continuously monitor the fluid flow parameters and send corresponding signals to recirculation pump (722). The recirculation pump (722) speed is altered based on the inputs from the sensor (760) to keep the fluid flow at desired rate. Recirculation pump (722) is used to exchange process fluid from bioreactor through hollow fibre filters (HFF) and back to bioreactor. This low shear pump is suitable for perfusion applications. Flow sensor (760) is provided on permate line to monitor the permeate flow rate. The recirculation pump (722) flow rate is also adjusted based on the permeate flow rate.

Filters (730) contain HFF membranes that are used to hold the cells and product based on perfusion applications. HFF can be switched automatically when primary HFF is clogged. Any clogging in the filters and reduction in flow is timely sensed by sensors (760) and corresponding signal is sent to adjust the flow from the recirculation pump (722).

Pneumatic pinch valves (762) are provided to divert the flow of process fluid based on HFF in use. These valves are automatically closed or opened based on process conditions like pressure of the fluid, clogging in the filters.

Steady state perfusion and cell bleed collection may be provided using different pumps and reservoirs. For example, the reservoir (750) is used for final harvest collection after batch termination. Reservoir (740) is used for permeate collection with auto switch option. Reservoirs (740) can be switched automatically if primary reservoir is filled. Reservoir (770) is used for cell bleed collection and accurately controlled using feedback from weighing scales. Pump (780) is used for cell bleed during perfusion cell culture to maintain steady state perfusion process and pump (742) is used to harvest permeate from HFF filter during the cell culture run.

Existing bioreactor systems require employing multiple pumps at different locations along the recirculation line and permeate line to control the process fluid flow. However, need of multiple pumps is obviated using the system and method according to the aspect of the present disclosure. Employing sensors (760) and generating a signal indicative of the fluid flow parameters to control the recirculation pump (722) facilitates use of a single recirculation pump (722). Use of several pumps to control process fluid flow is avoided and a compact system design is achieved.

According to further aspect of the present specification, a method (800) of controlling the fluid flow in a bioreactor system (700) is disclosed. The method (800) includes providing (805) a bioreactor system (700) including a bioreactor volume (720), a filtration part (730), and a recirculation line (721) between the bioreactor volume (720) and the filtration part (730), the recirculation line (721) including a recirculation pump (722). The method (800) further includes providing (810) a plurality of sensors (760) along the recirculation line (721) and monitoring the fluid flow parameters using the sensors (760). The method further includes sending (820) a plurality of signal from the sensors (760) indicative of the fluid flow parameters to controllers and controlling (830) the fluid flow rate at the recirculation pump (722). The method additionally includes employing (840) a plurality of valves (762) to control the flow of process fluid from the recirculation pump (722) based on the process conditions. Net flow from the bioreactor (720) is controlled and adjusted as function of weight of the bioreactor (720) and the fluid flow rate from the recirculation pump (722).

The system (700) and method (800) have several advantages over the existing systems. The method (800) is an automated and continuous process that enables reservoir switch among the different reservoirs (710). Provision of additional filter makes repairing and maintenance of filters during process run. Addition of a filter during a process run is possible due to provision of multiple filters. Filter mounting is outside the system as a separate attachment. This gives flexibility of attaching multiple filters of different size, without affecting system design. Bottom inlet to the perfusion system along with optimized tubing length provides for bubble trap region to minimize bubbles entry into the flowpath and ability to prime the entire flowpath along with the filter reduces the process time, manual intervention and cross contamination. The flow kit design is optimized for low cell shear and plug and play arrangement with XDR bioreactor is enabled.

While the disclosed embodiments of the subject matter described herein have been shown in the drawings and fully described above with particularity and detail in connection with several exemplary embodiments, it will be apparent to those of ordinary skill in the art that many modifications, changes, and omissions are possible without materially departing from the novel teachings, the principles and concepts set forth herein, and advantages of the subject matter recited in the appended claims. Hence, the proper scope of the disclosed innovations should be determined only by the broadest interpretation of the appended claims so as to encompass all such modifications, changes, and omissions. In addition, the order or sequence of any process or method steps may be varied or re-sequenced according to alternative embodiments.

1. A method for controlling fluid flow in a bioreactor system, the method comprising:
providing a bioreactor system including a bioreactor volume, a filtration part, and a recirculation line between the bioreactor volume and the filtration part, the recirculation line including a recirculation pump;
providing a plurality of sensors along the recirculation line and monitoring fluid flow or pressure parameters in the recirculation line using the sensors;
sending a plurality of signals from the sensors indicative of the fluid flow or pressure parameters to one or more controllers; and
controlling a fluid flow rate at the recirculation pump by means of the one or more controllers.

2. The method as claimed in claim 1, further comprising employing a plurality of valves to control flow of process fluid of the recirculation pump based on process conditions including fluid pressure and clogging in filters.

3. The method as claimed in claim 1, wherein net flow from the bioreactor is controlled and adjusted as function of weight of the bioreactor and the fluid flow rate of the recirculation pump.

4. The method as claimed in claim 1, wherein a single recirculation pump is used to exchange process fluid from bioreactor through hollow fiber filters (HFF) and back to bioreactor.

5. The method as claimed in claim 1, wherein the sensors are single use pressure sensors that monitor process pressure in the flowpath and tangential flow filtration (TFF) specific parameters, wherein the one or more controllers are configured to calculate transmembrane pressure (TMP) and pressure difference (Delta P) based on the single use pressure sensors output.

6. The method as claimed in claim 1, wherein a plurality of filters are employed along the recirculation line for continuous operation of the bioreactor system.

7. The method as claimed in claim 1, wherein a cell bleed in reservoir is controlled using a pump to maintain steady state perfusion.

8. The method as claimed in claim 1, further comprising providing a bottom inlet to the perfusion system to minimize bubble entry into the flowpath.

9. A control system for a bioreactor, the system comprising:
a plurality of media containers adapted to store reaction media;
at least one bioreactor fluidicly connected to the plurality of media containers;
at least one filter connected to the bioreactor via a recirculation line, the recirculation line including a recirculation pump in the recirculation line; and
a plurality of sensors for sensing fluid parameters in the recirculation line;
one or more controllers adapted to:
receive signals from the sensors indicative of the fluid parameters; and
send a control signal to the recirculation pump to control fluids in the recirculation line.

10. The control system as claimed in claim 9, further comprising a cell bleed tank system located on the recirculation line and positioned after a recirculation motor pump to improve cell viability and steady state of perfusion.

11. The control system as claimed in claim 9, further comprising a plurality of valves to control the flow of fluid from the recirculation pump based on process conditions including fluid pressure and filter clogging.

12. The control system as claimed in claim 9, further comprising a plurality of filters along the recirculation path for continuous operation of bioreactor system.

13. The control system as claimed in claim 9, wherein the sensors are single use pressure sensors that monitor process pressure in the flowpath and tangential flow filtration (TFF) specific parameters, wherein the one or more controllers are configured to calculate transmembrane pressure (TMP) and pressure difference (Delta P) based on the single use pressure sensors output.

14. The control system as claimed in claim 9, wherein a bottom inlet is provided to the perfusion system to minimize bubbles entry into the flowpath.
